(19) United States
(12) Patent Application Publication
(10) Pub. No.: US 2026/0243775 A1
(43) Pub. Date: Aug. 20, 2026

(54) CELL LABELING AND ANNOTATION IN MICRO-ORGANOSPHERES

(71) Applicant: Xilis, Inc., Durham, NC (US)

(72) Inventors: Zhaohui Wang, Durham, NC (US); Xiling Shen, Durham, NC (US); Eric Bankaitis, Durham, NC (US); Shaun Steele, Durham, NC (US)

(21) Appl. No.: 19/159,413

(22) PCT Filed: Mar. 1, 2024
(86) PCT No.: PCT/US2024/018019

§ 371 Date: Aug. 25, 2025

Related U.S. Application Data
(60) Provisional application No. 63/449,666, filed on Mar. 3, 2023.

(51) Int. Cl. G01N 33/58

(52) U.S. Cl. CPC G01N 33/582

(57) ABSTRACT

Methods and materials for labeling and annotating live cells in MicroOrganoSpheres (MOS), and methods for assessing MOS containing labeled live cells, are provided herein. The methods being applied for use in detecting treatments for diseases, particularly cancer, in mammals.

### CROSS-REFERENCE TO RELATED APPLICATIONS

This application claims benefit of priority from U.S. Provisional Application Ser. No. 63/449,666, filed Mar. 3, 2023. The disclosure of the prior application is considered part of (and is incorporated by reference in) the disclosure of this application.

### TECHNICAL FIELD

This document relates to methods and materials for labeling cells in three-dimensional (3D) Micro-OrganoSpheres (MOS), and to methods and materials for using MOS containing labeled live cells. For example, this document provides methods and materials for labeling specific types of cells (e.g., tumor cells, immune cells, fibroblasts, endothelial cells, or epithelial cells) in MOS, methods and materials for determining the effect of various therapeutic agents on the labeled cells in the MOS, and methods and materials for treating mammals based, at least in part, on the determined effects.

### BACKGROUND

Model cell and tissue systems are useful for biological and medical research. Immortalized cell lines can be derived from tissue and cultured in two-dimensional (2D) conditions (e.g., in Petri dishes or well plates), but 2D cell lines do not correlate well with individual patient response to therapies. In contrast, three-dimensional (3D) cell culture models (e.g., spheroids and organoids) can be particularly helpful in developmental biology, disease pathology, regenerative medicine, drug toxicity and efficacy testing, and personalized medicine. Multicellular tumor spheroids can be obtained by culturing cancer cell lines under non-adherent conditions. Spheroids typically form as freely floating cell aggregates in ultra-low attachment plates, and have been shown to maintain more stem cell associated properties than 2D cell culture. Organoids are in vitro derived cell aggregates that include a population of stem cells that can differentiate into cells of major lineages. Organoids typically have a diameter of more than one mm, and can be cultured through multiple passages. However, it typically takes several months to develop and test drug sensitivity in organoids, which can decrease clinical applicability because, ideally, an assay should be performed from a single core biopsy within about 7 to 10 days. In addition, the number of organoids obtained from a clinically relevant 18-gauge core biopsy generally is not sufficient to perform high throughput drug screens.

### SUMMARY

This document is based, at least in part, on the development of methods and materials for staining cells (e.g., live cells) in Micro-OrganoSpheres (MOS), and methods for using the stained MOS. For example, this document provides rapid live cell staining approaches that can be used for annotating cell types in MOS and/or for phenotypic screening of cells in MOS. The methods provided herein allow for real time observation of live cells in culture in MOS, without requiring fixing of the cells. In addition, the methods provided herein can be used, for example, to observe the behavior of cells within MOS, to track interactions between cells within MOS, and to assess the persistence of cell responses to drugs or other therapeutic agents. In some cases, the methods and materials provided herein include the use of patient-derived Micro-OrganoSpheres (PMOS) that contain cells originating from a mammal. For example, PMOS (also referred to herein simply as “MOS”) can contain cells extracted from a small patient biopsy, from resected patient tissue (e.g., resected primary tumor or tissue from a dysfunctional organ), and/or from established patient-derived models of cancer (PDMCs), including patient-derived xenografts (PDX) and organoids. In some cases, MOS can contain cells from a cell line (e.g., a genetically engineered cell line). The properties of MOS and the labeling of cells (e.g., live cells) therein can allow for interrogation of biological processes, including evaluation of various responses of the immune system to new and existing therapies.

An example of an assay described herein that includes the use of live cell staining in MOS is the detection and measurement of phagocytosis in 3D MOS. As demonstrated herein, for example, phagocytosis of labeled cells (e.g., tumor cells) by phagocytic cells initially included within or added to MOS generated from multiple myeloma patient (MM) samples was observed and assessed. In addition, PMOS generated from MM tissue were labeled to determine the effect of candidate agents (e.g., therapeutic agents such as drugs) on phagocytosis.

In general, one aspect of this document features methods that include labeling a population of cells within Micro-OrganoSpheres (MOS) with a detectable label, where at least a portion of the cells within the population comprise the detectable label. The labeling can include adding the detectable label to already formed MOS. The labeling can include adding the detectable label to the population of cells before the MOS are formed. The portion of cells can include cancer cells, immune cells, fibroblasts, endothelial cells, stromal cells, or epithelial cells. The MOS can include multiple myeloma cells. The detectable label can be a fluorescent dye. The detectable label can be a pH-sensitive label. The detectable label can be attached to the cells via an antibody or antibody fragment, where the antibody or antibody fragment has an antigen binding region that interacts with an epitope on the cells. The detectable label can be attached to the cells via an Fab antibody fragment. The epitope can be CD38, CD138, CD33, CS1, BCMA, CD45, cell adhesion molecule, EpCAM, TROP2, CD3, CD8, PD-1, CD-31, CD19, FAPalpha, ASGR1, or HER2. The label can be a pH-sensitive label, and the method can further include measuring the level of signal over time to determine a rate or extent of phagocytosis. The label can be a pH-sensitive label, and the method can further include detecting the presence or absence of a signal output from the pH-sensitive detectable label, where the presence of the signal indicates phagocytosis of cells within the population, and where the absence of the signal indicates a lack of phagocytosis of cells within the population.

In another aspect, this document features a method that includes, or consists essentially of, providing MOS comprising a population of cells, where at least a portion of the cells within the population comprise a pH-sensitive detectable label, measuring a first level of signal from the pH-sensitive detectable label, where the first level of signal is directly correlated to a level of phagocytosis of the cells within the portion of cells, contacting the MOS with a candidate agent, measuring a second level of signal from the pH-sensitive detectable label, where the second level of signal is directly correlated to a level of phagocytosis of cells within the portion of cells after the contacting, and comparing the first measured level of signal to the second measured level of signal and, when the second measured level of signal is increased compared to the first measured level of signal, identifying the candidate agent as being a phagocytosis-enhancing agent for the population of cells. The portion of cells can include cancer cells. The MOS can include multiple myeloma cells. The pH-sensitive label can be a fluorescent dye. The pH-sensitive detectable label can be attached to the cells via an antibody or antibody fragment, where the antibody or antibody fragment has an antigen binding region that interacts with an epitope on the cells. The pH-sensitive detectable label can be attached to the cells via an Fab antibody fragment. The epitope can be CD38, CD138, CD33, CS1, BCMA, CD45, cell adhesion molecule, EpCAM, TROP2, CD3, CD8, PD-1, CD-31, CD19, FAPalpha, ASGR1, or HER2. The method can further include measuring the level of signal over time to determine a rate of phagocytosis.

In another aspect, this document features a method for treating a mammal. The method can include, or consist essentially of, providing MOS comprising a population of cells from the mammal, where at least a portion of the cells within the population comprise a pH-sensitive detectable label; measuring a first level of signal from the pH-sensitive detectable label, where the first level of signal is directly correlated to a level of phagocytosis of the cells within the portion of cells; contacting the MOS with a candidate agent; measuring a second level of signal from the pH-sensitive detectable label, where the second level of signal is directly correlated to a level of phagocytosis of cells within the portion of cells after the contacting; determining that the second measured level of signal is increased compared to the first measured level of signal; and administering the candidate agent to the mammal. The plurality of cells can include cancer cells. The MOS can include multiple myeloma cells. The pH-sensitive label can be a fluorescent dye. The pH-sensitive detectable label can be attached to the cells via an antibody or antibody fragment, where the antibody or antibody fragment has an antigen binding region that interacts with an epitope on the cells. The pH-sensitive detectable label can be attached to the cells via an Fab antibody fragment. The epitope can be CD38, CD138, CD33, CS1, BCMA, CD45, cell adhesion molecule, EpCAM, TROP2, CD3, CD8, PD-1, CD-31, CD19, FAPalpha, ASGR1, or HER2. The method can further include measuring the level of signal over time to determine a rate of phagocytosis.

In still another aspect, this document features a method for treating a mammal, where the method includes, or consists essentially of, administering a therapeutic agent to a mammal identified as having cells that undergo an increased level of phagocytosis when contacted with the therapeutic agent, where the mammal was identified by providing MOS comprising a population of cells from the mammal, where at least a portion of the cells within the population comprise a pH-sensitive detectable label; measuring a first level of signal from the pH-sensitive detectable label, where the first level of signal is directly correlated to a level of phagocytosis of the cells within the portion of cells; contacting the MOS with a candidate agent; measuring a second level of signal from the pH-sensitive detectable label, where the second level of signal is directly correlated to a level of phagocytosis of cells within the portion of cells after the contacting; and determining that the second measured level of signal is increased compared to the first measured level of signal. The plurality of cells can include cancer cells. The MOS can include multiple myeloma cells. The pH-sensitive label can be a fluorescent dye. The pH-sensitive detectable label can be attached to the cells via an antibody or antibody fragment, where the antibody or antibody fragment has an antigen binding region that interacts with an epitope on the cells. The pH-sensitive detectable label can be attached to the cells via an Fab antibody fragment. The epitope can be CD38, CD138, CD33, CS1, BCMA, CD45, cell adhesion molecule, EpCAM, TROP2, CD3, CD8, PD-1, CD-31, CD19, FAPalpha, ASGR1, or HER2. The method can further include measuring the level of signal over time to determine a rate of phagocytosis.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1D demonstrate the establishment of live MOS staining technology. FIG. 1A illustrates the workflow of a live MOS staining approach. FIG. 1B includes representative epithelial cell adhesion molecule (EpCAM)/CD45 staining images of MOS that encapsulated different ratios of PBMC and colorectal cancer (CRC) cells. Magenta indicates EpCAM staining, while green indicates CD45 staining. FIG. 1C includes representative images showing live cell staining of MOSs that encapsulate different types of cells or primary tissue-derived cells. Top left, MOS that encapsulated CRC and tumor infiltrated lymphocytes, stained with EpCAM and CD45; top center, MOS derived from primary lung tissue, stained with EpCAM and CD45; top right, mixed culture MOS that encapsulated cancer-associated fibroblasts or CRC cells, stained with FAPalpha and EpCAM; bottom left, MOS that encapsulated CRC and hepatocytes, stained with EpCAM and ASGR1; bottom center, MOS that encapsulated CRC cells, HUVEC, and cancer-associated fibroblasts, stained with CD31 and EpCAM; bottom right, MOS that encapsulated primary lung-derived cells, stained with EpCAM and CD45. FIG. 1D includes representative images showing the duration of EpCAM staining signals in CRC MOS culture over the course of 7 days.

FIGS. 2A-2G show that EpCAM staining of live MOS does not affect cell growth or drug responses of the MOS. FIGS. 2A-2C are graphs plotting growth rates of three CRC MOS cultures (501299, 500040, and 501304, respectively), with or without EpCAM staining. FIGS. 2D-2G are graphs plotting the viability of CRC MOS after treatment with the chemotherapeutic drug, 5-fluorouracil (5-FU) with or without EpCAM staining.

FIGS. 3A-3C show that live MOS staining delineates the tumor cell response to anticancer treatment in MOS that encapsulate CRCs in combination with non-cancer cells (fibroblasts, endothelial cells, and PBMCs). FIG. 3A includes representative images showing live MOS staining of CRC cells co-encapsulated with fibroblasts, endothelial cells, and PBMCs, stained with EpCAM and treated with vehicle or oxaliplatin for three days, followed by staining with Calcein AM (CAM) and ethidium homodimer (EtH). FIG. 3B is a graph plotting differential drug responses observed for the CRC only condition and for CRCs co-encapsulated with the non-cancer cells, measured by CTG assay. FIG. 3C is a graph plotting drug responses of the CRC only condition and the co-encapsulated group, indicating that they were similar based on EpCAM staining and live dead cell imaging analysis.

FIG. 4A is a schematic illustrating the steps in a representative assay using PHRODO™-labeled CD38 antibody to tag MM myeloid cells, resulting in generation of a signal (in this case, green fluorescence) that can be detected by flow cytometry when the myeloid cells are phagocytosed by macrophages. The steps in the illustrated method include (1) labeling CD38 antibodies with PHRODO™; (2) adding a PHRODO™ labeled CD38 antibody into MM cell culture to tag CD38⁺ myeloid cells; and (3) mixing the PHRODO™-tagged MM cells with or without PBMC-derived macrophages, to generate MOS. Co-culture with macrophages derived from monocytes (MDMs) can be used to ensure that phagocytosis is occurring. FIG. 4B is a schematic illustrating the steps in a representative assay using Zymosan to assess phagocytosis. Zymosan is a glucan with repeating glucose units connected by β-1,3-glycosidic linkages, and can be taken up by macrophages. Labeling Zymosan can provide a measure of total phagocytosis, which can serve as a control. The steps in the illustrated method include (1) labeling Zymosan with PHRODO™ green, (2) co-packaging PHRODO™-labeled Zymosan and a MM sample into MOS, and (3) using flow cytometry to determine whether phagocytosis is occurring, in which case the PHRODO™-labeled Zymosan will become green and can be detected by flow cytometry. FIG. 4C is a chart showing assay readouts that can be conducted for the methods depicted in FIGS. 4A and 4B. In particular, at Day 1 of MOS culture (about 24 hours after MOS generation) and Day 5 of MOS culture, flow cytometry can be carried out to measure cells labeled with fluorescein isothiocyanate (FITC), CD11b (a marker for myeloid cells), and CD16 (a marker for natural killer cells, neutrophils, monocytes, macrophages, and certain T cells). In addition, on Day 5, the MOS can be imaged.

FIG. 5A includes representative images of MM MOS cultured with Zymosan (top) or PHRODO™-labeled anti-CD38 antibodies (bottom) as depicted in FIG. 4B and 4A, respectively. FIG. 5B shows histograms generated by flow cytometry for MM MOS with no labeling (top), PHRODO™-labeled anti-CD38 antibodies (middle), or PHRODO™-labeled Zymosan (bottom).

FIGS. 6A and 6B plotting the percentage of phagocytes (FIG. 6A) or CD11b⁺ phagocytes (FIG. 6B) in the indicated samples. PHRODO™-based phagocytosis assays were performed as described for FIGS. 4A and 4B. Data were collected after 24 hours for (1) unlabeled MM samples (without PHRODO™-labeled anti-CD38 antibodies), (2) PHRODO™-labeled MM samples, (3) Zymosan-labeled MM samples that were co-packaged with PHRODO™-labeled Zymosan, and (4) MM samples with MDM (PBMC-derived macrophages that were co-packaged with MM samples). FIG. 6A is a graph plotting % green cells in live cells for each condition after 24 hours of incubation. FIG. 6B is a graph plotting gating of CD11b⁺ cells from the green cells of FIG. 6A.

FIG. 7 is a graph plotting the percentage of PHRODO™+CD11b+cells 4 hours after treatment with IgG4 or anti-CD47, or with no treatment.

FIGS. 8A and 8B are graphs plotting the percent PHRODO™⁺ cells (FIG. 8A) and percentage of phagocytosis (FIG. 8B) in the indicated samples. All data were generated by flow cytometry from a second frozen MM sample. Frozen thawed MM samples were tagged with PHRODO™-labeled antibodies, and MOS were generated. Phagocytosis assays were performed on Day 0 and on Day 5 of MOS culture. Data were collected 24 hours later. The conditions used were: (1) no PHRODO™: no PHRODO™-labeled antibody added; (2) Zymosan: MM sample co-packaged with PHRODO™-labeled Zymosan (data were only collected on Day 1); and (3) untreated: vehicle control. FIG. 8A is a graph plotting the % green cells in live cells for each condition after 24 hours of incubation. FIG. 8B is a graph plotting gating of CD11b⁺ cells from the green cells of FIG. 8A.

FIG. 9A is a schematic illustrating the steps in a representative time course assay in which PHRODO™-labeled CD38 antibody was used to tag MM myeloid cells in MOS, and an anti-CD47 antibody was used to treat the MOS, followed by flow cytometry at the indicated time points. FIG. 9B is a graph plotting the results of a time course assay, plotting the percentage of CD11b⁺ cells that were PHRODO™⁺ in MOS that were stained with PHRODO™-anti-CD38, isotype PHRODO™, or unstained.

### DETAILED DESCRIPTION

### Methods for Generating MOS

This document provides methods and materials for staining live cells in MOS, and methods for using the live cell-stained MOS. For example, the live cell staining methods described herein can be used to annotate cell types in MOS, to conduct phenotypic screening of cells in MOS, to observe live cells in MOS in real time, to track the behavior of cells within MOS, to observe interactions between cells within MOS, and/or to assess cell responses to drugs or other therapeutic agents. In some cases, the methods and materials provided herein include the use of PMOS that contain cells originating from a mammal, such as cells extracted from a patient biopsy, cells obtained from resected patient tissue (e.g., resected primary tumor or tissue from a dysfunctional organ), and/or cells from established PDMCs, PDXs, and organoids. The labeling of cell in MOS can allow for interrogation of biological processes, including evaluation of various responses of the immune system to new and existing therapies.

In general, the methods and materials provided herein relate to an antibody-based staining approach for labeling live cells in MOS droplets, which can allow for MOS-Fluorescent Antibody Cell Tracking (MOS-FACT). As described herein, antibodies coupled to fluorescent labels can efficiently diffuse into MOS droplets and can stain surface markers of target cells. Thus, using fluorescent conjugated primary antibodies targeted to specific cell surface markers, different type of cells (e.g., tumor cells, immune cells, fibroblasts, endothelial cells, cholangiocytes, hepatocytes, etc.) in the MOS droplets can be specifically labeled and annotated, generally within 1 to 4 hours of staining, without fixing or killing the cells. The live MOS staining signals also can reflect and inform the histological diagnosis of primary tissue. As described herein, the antibody fluorescence signals can last up to 7 days, and antibody staining of cells in MOS does not affect the drug responses of MOS treated with chemotherapeutic drugs or tyrosine kinase inhibitors (TKIs). In combination with imaging (e.g., high content confocal imaging), the approaches described herein enable the longitudinal tracking of cells and phenotypic screening in MOS culture. The MOS are not adherent to culture surfaces, but rather are suspended in their culture media, and therefore the MOS are pipettable and can readily be transferred to other vessels, containers, or devices. Without being bound by a particular mechanism of action, this characteristic, in combination with the stability of the fluorescent labels over time and the fact that the labeled cells remain alive in the MOS, means that the analysis of a population of labeled MOS is not limited to imaging, but rather can include downstream analyses such as flow cytometry, single cell RNA sequencing, high throughput analytics, etc.

Methods for making MOS can employ, for example, techniques of cell biology, cell culture, molecular biology, transgenic biology, microbiology, recombinant DNA, and immunology. Examples of such techniques are described elsewhere. See, e.g., Molecular Cloning A Laboratory Manual (1989), 2^{nd} Ed., Sambrook, Fritsch, and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, Gait, ed., 1984; Nucleic Acid Hybridization, Hames and Higgins, eds., 1984; Transcription and Translation, Hames and Higgins, eds., 1984; Culture of Animal Cells, Freshney, Alan R. Liss, Inc., 1987; Immobilized Cells And Enzymes, IRL Press, 1986; Perbal (1984), A Practical Guide To Molecular Cloning: See Methods In Enzymology (Academic Press, Inc., N.Y.); Gene Transfer Vectors For Mammalian Cells, Miller and Calos, eds., Cold Spring Harbor Laboratory, 1987; Methods in Enzymology, Vols. 154 and 155, Wu et al., eds., Academic Press Inc., N.Y. ; Immunochemical Methods in Cell and Molecular Biology, Mayer and Walker, eds., Academic Press, London, 1987; and Handbook of Experimental Immunology, Volumes I-IV, Weir and Blackwell, eds., 1986.

MOS can be formed from primary cells that are normal (e.g., normal organ tissue) or from tumor tissue. For example, in some cases, PMOS can be formed from cancerous tumor biopsy tissue, enabling tailored treatments that can be selected using the particular tumor tissue examined. Methods for generating MOS as described herein permit the formation of hundreds, thousands or even tens of thousands (e.g., 500, 750, 1000, 2000, 5000, 10,000 or more) MOS from a single tissue biopsy, within a few hours of the biopsy being removed from the patient. In general, dissociated primary cells from a patient biopsy can be combined with a fluid matrix material, such as a substrate basement membrane matrix (e.g., MATRIGEL®, a solubilized basement membrane matrix secreted by Engelbreth-Holm-Swarm mouse sarcoma cells, available from Sigma), to form MOS. The resulting plurality of MOS can have a predefined range of sizes (e.g., diameters from about 10 μm to about 700 μm, and any sub-range therewithin), and initial number of primary cells (e.g., from about 1 to about 1000, and in particular lower numbers of cells, such as form about 1 to about 200). The number of cells and/or the diameter can be controlled within, e.g., ±5%, ±10%, ±15%, ±20%, ±25%, ±30%, etc. The PMOS can have a very high survival rate (e.g., >75%, >80%, >85%, >90%, >95%), and can be stable for use and testing within a very short period of time, generally within the first 1 to 14 days after being formed (e.g., within 1 day, within 2 days, within 3 days, within 4 days, within 5 days, within 6 days, within 7 days, within 8 days, within 9 days, within 10 days, within 11 days, within 12 days, within 13 days, or within 14 days). This can allow for rapid testing on a potentially huge number of patient-specific and biologically relevant PMOS, which may save critical time when developing and deploying a patient therapy such as a cancer treatment plan. The PMOS can rapidly form 3D cellular structures that replicate and correspond to the tissue environment from which they were biopsied, such as a 3D tumor microenvironment. In some cases, the PMOS can be referred to as “droplets.” Each PMOS can include (e.g., as part of the fluid matrix material) growth factors and structural proteins (e.g., collagen, laminin, nidogen, etc.) that can mimic the original tissue (e.g., tumor) environment.

In some cases, the MOS used in the methods provided herein can contain cancer cells as well as one or more other types of cells (e.g., fibroblasts, endothelial cells, and/or immune cells, including cells that can carry out phagocytosis, such as macrophages, neutrophils, monocytes, dendritic cells, and/or osteoclasts). MOS can be derived from, for example, bone marrow tissue samples from mammals having multiple myeloma (MM) (referred to herein as “MM MOS”), from acute myeloid leukemia (AML) bone marrow samples, from a solid tumor sample (e.g., a biopsy from a colorectal tumor, a breast tumor, a kidney tumor, a liver tumor, a lung tumor, an ovarian tumor, or a prostate tumor), or from any other appropriate type of sample. In some cases, MOS can contain cells from a cell line (e.g., a genetically engineered cell line). Methods and materials for generating genetically engineered cell lines (e.g., using CRISPR components) are described in, for example, PCT Publication No. WO2024/040145, which is incorporated herein by reference in its entirety. In some cases, MOS can contain no cells from a cell line.

MOS that can be labeled and used in the methods provided herein typically are spheres formed from dissociated cells (e.g., dissociated primary cells distributed within biopsy material). The MOS can have a diameter of about 50 μm and about 500 μm (e.g., from about 50 μm to about 400 μm, from about 50 μm to about 300 μm, from about 50 μm to about 250 μm, from about 100 μm to about 500 μm, from about 100 μm to about 250 μm, or from about 50 μm to about 200 μm). The MOS can initially contain from about 1 to about 1000 dissociated primary cells distributed within the base material (e.g., from about 1 to about 750, from about 1 to about 500, from about 1 to about 400, from about 1 to about 300, from about 1 to about 200, from about 1 to about 150, from about 1 to about 100, from about 25 to about 200, from about 50 to about 150, or from about 25 to 100 primary cells).

Despite their small size (often from about 50 to about 250 μm) and low cell density (e.g., often from about 50 to about 200 cells per MOS), the MOS can be used immediately or cultured for a relatively short period of time (e.g., 14 days or less, 10 days or less, 7 days or less, or 5 days or less). Moreover, the cells within the MOS can survive while maintaining most or even all of the characteristics of the tissue from which they were extracted. The survival rate of the cells within the MOS typically is high, and the MOS can be cultured for days or even weeks, through multiple passages in which the cells can divide, cluster, and form structures similar to the parent tissue. Also, in some cases, cells from the dissociated tissue from which the MOS were generated can form morphological structures inside even the smallest MOS. Although in some applications the presence of such structures is not necessary for the utility of these MOS (such as when they are used before substantial structural reorganization has occurred), in some cases such structures can be particularly useful.

Any appropriate method can be used to generate MOS, including the methods described herein. The following definitions are set forth herein to facilitate explanation of the presently disclosed subject matter.

The term “an unpolymerized mixture” is used herein to refer to a composition containing biologically-relevant materials, including a dissociated tissue sample and a first fluid matrix material. The fluid matrix material typically is a material that can be polymerized to form a support or support network for the dissociated tissue and/or cells dispersed within it. Once polymerized, the polymerized material may form a hydrogel and may be formed and/or may include proteins forming the biocompatible medium, in addition to the cells. A biocompatible medium suitable for use in the methods disclosed herein can be formed from any biocompatible material that is a gel, a semi-solid, or a liquid, such as a low-viscosity liquid, at room temperature (e.g., 25° C.), and can be used as a three-dimensional substrate for cells, tissues, proteins, and other biological materials of interest. Exemplary materials that can be used to form a biocompatible medium in accordance with the presently-disclosed subject matter include, but are not limited to, polymers and hydrogels containing collagen, fibrin, chitosan, MATRIGEL® (BD Biosciences, San Jose, CA), polyethylene glycol (PEG), dextrans (e.g., chemically crosslinkable or photo-crosslinkable dextrans), and the like, as well as electrospun biological, synthetic, or biological-synthetic blends. In some cases, the biocompatible medium can be a hydrogel.

The term “hydrogel” is used herein to refer to two-or multi-component gels having a three-dimensional network of polymer chains, where water acts as the dispersion medium and fills the space between the polymer chains. Hydrogels used in accordance with the presently-disclosed subject matter generally can be chosen for a particular application based on the intended use of the structure, taking into account the parameters that are to be used to form the MOS, as well as the effect the selected hydrogel will have on the behavior and activity of the biological materials (e.g., cells) incorporated into the biological suspensions that are to be placed in the structure. Exemplary hydrogels for use with the presently-disclosed subject matter include polymeric materials such as, without limitation, alginate, collagen (including collagen types I and VI), elastin, keratin, fibronectin, proteoglycans, glycoproteins, polylactide, polyethylene glycol, polycaprolactone, polycolide, polydioxanone, polyacrylates, polyurethanes, polysulfones, peptide sequences, proteins and derivatives, oligopeptides, gelatin, elastin, fibrin, laminin, polymethacrylates, polyacetates, polyesters, polyamides, polycarbonates, polyanhydrides, polyamino acids carbohydrates, polysaccharides and modified polysaccharides, and derivatives and copolymers thereof as well as inorganic materials such as glass such as bioactive glass, ceramic, silica, alumina, calcite, hydroxyapatite, calcium phosphate, bone, and any combination thereof.

With further regard to the hydrogels used to produce MOS, in some cases, the hydrogel can contain a material selected from the group consisting of agarose, alginate, collagen type I, a polyoxyethylene-polyoxypropylene block copolymer (e.g., PLURONIC® F127; BASF Corporation, Mount Olive, NJ), silicone, polysaccharide, polyethylene glycol, and polyurethane. In some cases, the hydrogel can be made up of alginate.

In some cases, MOS can contain biologically-relevant materials. The phrase “biologically-relevant materials” refers to materials that are capable of being included in a biocompatible medium as defined herein and subsequently interacting with and/or influencing biological systems. For example, biologically-relevant materials can be magnetic beads (such as beads that are magnetic themselves or that contain a material that responds to a magnetic field, such as iron particles) that can be combined as part of the unpolymerized material to aid in the production of MOS (e.g., for the separation and purification of MOS). As another example, in some cases, biologically-relevant materials can include cells in addition to the dissociated tissue sample (e.g., biopsy material). In the unpolymerized mixture, the dissociated tissue sample and the additional biologically relevant material can be present as a uniform mixture or as a distributed mixture (e.g., on one half or another portion of the MOS, such as just in the core or just in the outer region of the formed MOS). In some cases, the additional biologically-relevant material within the unpolymerized material can be suspended with the dissociated tissue sample in suspension (e.g., prior to polymerization of the droplet forming the MOS). In some cases, the biologically-relevant material that optionally can be included with the dissociated tissue sample (e.g., biopsy) may contain a number of cell types, including preadipocytes, mesenchymal stem cells (MSCs), endothelial progenitor cells, T cells, B cells, mast cells, and/or adipose tissue macrophages, MDMs, as well as small blood vessels or microvascular fragments found within the stromal vascular fraction.

As used herein, the term “passaged” refers to the average number of doublings of the cells within the MM MOS. Although traditional passage number refers to the transfer or subculture of cells from one culture vessel to another, the cells within a MOS may be stably retained within the same MOS, and may continue to grow and divide. Thus, the passage number as used herein typically refers to the average number of doublings undergone by the dissociated cells from the biopsied tissue within the MOS. The population doubling number is the approximate number of doublings that the cell population has undergone since isolation (e.g., since forming of the MOS from the freshly dissociated biopsy tissue). In general, the MOS described herein can be cultured for a short period of time relative to the growth (e.g., doublings) of some or all of cells within the MOS (e.g., fewer than 10 passages, fewer than 9 passages, fewer than 8 passages, fewer than 7 passages, fewer than 6 passages, fewer than 5 passages, fewer than 4 passages, or fewer than 3 passages).

In some cases, the tissue sample (and cells contained therein) used to generate MOS can be taken from a mammal. The tissue sample can be from any appropriate mammal (e.g., a human or other mammal having MM, such as a dog, cat, mouse, rat, or rabbit), typically taken by biopsy. The tissue can be derived from, without limitation, a biopsy, a surgical specimen, an aspiration, a drainage, or a cell-containing fluid. Typically, MOS will contain multiple cell types that are resident in the tissue of origin, although it is also possible to form MOS from one specific cell type. The cells can be obtained directly from the subject without intermediate steps of subculture, or the cells can first undergo an intermediate culturing step to produce a primary culture. Any suitable methods for harvesting cells from biological tissues and/or cell containing fluids can be used. For example, suitable techniques used to obtain cells from biological tissues include those described by Mahesparan, Acta Neuropathol (1999) 97:231-239.

The cells in the tissue sample typically are first dissociated or separated from each other before forming the MOS. Dissociation of cells can be accomplished using any suitable method. For example, the cells can be treated mechanically and/or chemically, such as by treatment with enzymes. “Mechanical” dissociation typically includes disrupting connections between associated cells using, for example, a scalpel, scissors, or a machine such as an homogenizer. “Enzymatic” dissociation can include treating the cells with one or more enzymes to disrupt connections between associated cells (e.g., using a collagenase, dispase, DNAse, and/or hyaluronidase). One or more enzymes can be used under any suitable reaction conditions, such as incubation at 37° C. in a water bath or at room temperature.

The dissociated tissue can be treated to remove dead/dying cells and/or cell debris. The removal of such dead and/or dying cells can be accomplished using any appropriate means, such as beads and/or antibody methods. For example, since phosphatidylserine can be redistributed from the inner to outer plasma membrane leaflet in apoptotic or dead cells, the use of Annexin V-Biotin binding followed by binding of the biotin to streptavidin magnetic beads can enable the separation of apoptotic cells from living cells. Removal of cell debris can be achieved by techniques such as, for example, filtration.

The dissociated cells can be suspended in a carrier material prior to being combined with a fluid matrix material. Alternatively, the fluid matrix material can be referred to as a carrier material. In some cases, the carrier material can be a material that has a viscosity level that delays sedimentation of cells in a cell suspension, prior to polymerization and formation of the MOS. In such cases, the carrier material can have sufficient viscosity to allow the dissociated biopsy tissue cells to remain suspended in the suspension until polymerization. The viscosity required to achieve this can be optimized by monitoring the sedimentation rate at various viscosities and selecting a viscosity that gives an appropriate sedimentation rate for the expected time delay between loading the cell suspension into the apparatus that will form the MOS by polymerizing droplets of the unpolymerized material that includes the cells. In some cases, the unpolymerized material can be flowed or agitated by the apparatus (e.g., when lower viscosity materials are used), to keep the cells in suspension and/or distributed as desired.

As discussed above, in some cases the unpolymerized mixture that includes the dissociated tissue sample and the fluid matrix material can contain one or more other components, such as one or more biologically-relevant materials. Biologically-relevant materials that can be contained in an unpolymerized mixture include, without limitation, one or more of an extracellular matrix protein (e.g., fibronectin), a drug (e.g., a small molecule), a peptide, an antibody (e.g., to modulate any of cell survival, proliferation, or differentiation), and/or an inhibitor of a particular cellular function. Such biologically-relevant materials may be used, for example, to increase cell viability by reducing cell death and/or activation of cell growth/replication or to otherwise mimic the in vivo environment. The biologically-relevant material can include or may mimic one or more of the following components: serum, interleukins, chemokines, growth factors, glucose, physiological salts, amino acids, and hormones. The biologically-relevant material can, in some cases, supplement one or more agents in the fluid matrix material. In some cases, the fluid matrix material can be a synthetic gel (hydrogel), and can be supplemented with one or more biologically-relevant materials. In some cases, the fluid matrix can be a natural gel. Thus, the gel may include one or more extracellular matrix components such as collagen, fibrinogen, laminin, fibronectin, vitronectin, hyaluronic acid, fibrin, alginate, agarose, and/or chitosan. For example, MATRIGEL® includes bioactive polymers that are important for cell viability, proliferation, development, and migration. In some cases, the matrix material can be a gel that includes collagen type 1, such as collagen type 1 obtained from rat tails. The gel can be a pure collagen type 1 gel or can be one that contains collagen type 1 in addition to other components, such as other extracellular matrix proteins. In some cases, the fluid matrix can be a synthetic gel that does not occur in nature. Examples of synthetic gels include gels derived from polyethylene glycol (PEG), polyhydroxyethyl methacrylate (PHEMA), polyvinyl alcohol (PVA), or poly ethylene oxide (PEO). In some cases, the matrix material can include natural polymers, such as one or more of alginate, agarose, hyaluronic acid, collagen, gelatin, fibrin, and elastin, or can include a synthetic polymer, such as one or more of PEG and polyacrylamide. Both organic and inorganic synthetic polymers can be used.

MOS generally include a dissociated biopsy tissue (e.g., cells) in a fixed or known number or concentration of cells (e.g., cells/ml or cells/mm³) within the MOS. In some cases, the number of cells initially included in the MOS can be from 1 cell up to several hundred cells. In particular, for use in some assays (e.g., drug toxicity assays), it may be beneficial to generate MOS to include about 1 to about 75 cells, or about 1 to about 50 cells (that is, relatively lower numbers of cells). The number of cells per MOS can be set or selected by the technician generating the MOS. In some cases, an apparatus for generating MOS can include one or more controls to set the number of cells from the primary tissue that will be included in each MOS. The number of cells can be chosen or set based on how the MOS are intended to be used. For example, MOS having very low number of cells (e.g., 1 cell per MOS or 1 to 5 cells per MOS) can be particularly suitable for studying clonal diversity (e.g., for tumor heterogeneity). In some cases, each MOS grows from a single cell, one in which case can observe which clones are drug resistant, and these specific MOS can then be examined (e.g., by genomic sequencing) to determine the genomic (mutation) diversity related to the particular clone. A low to moderate number of cells per MOS (e.g., about 3 to about 30 cells, about 5 to about 30 cells, about 5 to about 25 cells, about 5 to about 20 cells, or about 10 to about 25 cells) can be particularly useful for rapid drug testing, including toxicity testing since these MOS typically grow quickly. A larger number of cells per MOS (e.g., about 20 to about 100 cells, about 30 to about 100 cells, about 40 to about 100 cells, or more than 50 cells) can be particularly suitable for mimicking tissue composition in each MOS, as the MOS may contain different lineages—potentially including epithelial (e.g., cancer) and mesenchymal (e.g., stromal, immune, or blood vessel) cells. It is noted that for MM MOS, including a relatively large number of cells per MOS (e.g., about 50 to about 200 cells per MOS) can provide each MOS with different cell types, which can act to maintain the MM cells.

In some cases, MOS can be produced by forming a droplet of an unpolymerized mixture (in some cases, a chilled mixture) of a dissociated tissue sample and a fluid matrix material in an immiscible material, such as a fluid hydrophobic material (e.g., oil). For example, a MOS may be formed by combining a stream of unpolymerized material with one or more streams of the immiscible material to form a droplet. The density of the cells present in the droplet can be determined by dilution of the dissociated material (e.g., cells) in the unpolymerized material. The size of the MOS may correlate to the size of the droplet formed. In general, the MOS is a spherical structure having a stable geometry.

MOS can have any appropriate size, which may be matched to the number of cells to be included. For example, the size of a MOA may be relatively small, having a diameter of about 20 μm to about 500 μm (e.g., about 50 μm or about 100 μm on average, or about 100 μm to about 200 μm). In some cases, the size of a MOS can be about 300 μm, in which case about 10 to about 50 cells (e.g., about 10 to about 30 cells) can be included in each MOS. The number of cells and the size can be varied and/or may be controlled. In some cases, the number of cells and/or the size of the MOS can be set by one or more controls on the apparatus used to forming the MOS. For example, the size of the MOS and/or the density of cells within the MOS can be adjusted by adjusting the flow rates and/or the concentration of the dissociated tissue sample (e.g., cells from a biopsy).

During culturing, the cells from the dissociated, biopsied tissue in the MOS can aggregate, cluster, or assemble within the MOS. Aggregates of cells can be highly organized, and may form defined morphology or may be a mass of cells that have clustered or adhered together. The organization may reflect the tissue of origin. In some cases, MOS can contain a single cell type (homotypic), while in other cases, the MOS can contain more than one cell type (heterotypic).

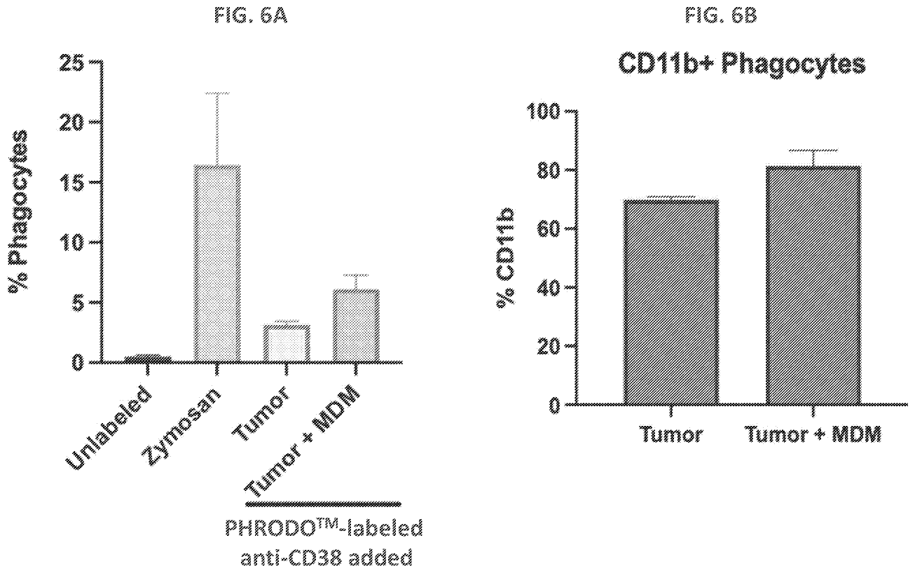

Additional description of methods and apparatuses for making MOS can be found in U.S. Patent Publication No. 2021-0285054. See, for example, paragraphs [0165] to [0183] and FIGS. 6-10 of U.S. Patent Publication No. 2021-0285054.

In some cases, MOS can be generated from a MM tissue sample. It is generally difficult to use MM tissue to replicate tumor cells, because MM cells typically are difficult to work with, and culturing MM cells in 3D culture can be challenging. Moreover, without bone marrow stromal cells, MM cells cannot survive. As described in Examples 1-3 of U.S. Provisional Application No. 63/404,472 (incorporated herein by reference in its entirety), however, MOS can be generated from MM biopsies, which was surprising given the above-referenced challenges. The technology described therein and herein can provide a tumor microenvironment for MM cells, allowing the MM cells to grow. Cells within the MM immune microenvironment (e.g., macrophages) also are captured in the MOS, expanding the range of drugs against which the MOS can be screened. This range includes, without limitation, agents that can modulate (e.g., drugs that can induce) phagocytosis by macrophages and other cells within the MM MOS.

In some cases, therefore, the tissue (e.g., biopsy) sample used to generate MOS (e.g., the dissociated tissue) can be derived from bone marrow obtained from a mammal having MM. In such cases, the tissue used in the MOS can include cells of the immune system, such as T lymphocytes, B lymphocytes, polymorphonuclear leukocytes, macrophages, and dendritic cells. The cells can include stem cells, progenitor cells, or somatic cells. These tissues and the resulting dissociated cells can be primary cells taken from a patient biopsy (e.g., by a needle biopsy). The dissociated cells can be incorporated into MM MOS, and can include cells that are commonly found in bone marrow. In that regard, exemplary cells that may be incorporated into MM MOS include plasma cells (normal plasma cells and cancerous plasma cells), stromal cells, hematopoietic stem cells, monocytes, macrophages, neutrophils, basophils, eosinophils, erythrocytes, dendritic cells, T cells, B cells, and natural killer (NK) cells. The tissue can be dissociated using any suitable technique.

As described in U.S. Provisional Application No. 63/404,472, in some cases, the preparation and use of MOS generated from MM bone marrow biopsies can be facilitated by (1) using about 50 to about 150 cells (e.g., about 50 to 100 cells, about 75 to about 125 cells, about 100 to about 150 cells, or about 100 cells) per MOS, (2) increasing the concentration of added MATRIGEL® to about 80-100% (e.g., about 80 to about 85%, about 85 to about 90%, about 90 to about 95%, about 95 to about 100%, about 80 to about 90%, or about 90 to about 100%), and/or (3) using ultra low binding plates. Without being bound by a particular mechanism, using relatively higher numbers of cells can increase the likelihood that the full complement of cells from the bone marrow microenvironment (e.g., stromal cells and immune cells) will be included in the MM MOS, which can be important from a drug screening standpoint. In addition, without being bound by a particular mechanism, using MATRIGEL® at a higher concentration can help to retain the MM cells within the MOS, and using low or ultra-low binding plates can reduce the likelihood that the MOS will attach to the bottom of the culture well. In addition, in some cases, the methods disclosed herein for making MM MOS can include adding a small amount of MATRIGEL® (e.g., 1%) to the culture medium rather than to the MOS mix, and/or adding one or more cytokines (e.g., IL-6, GM-CSF, IL-2, B cell activating factor (BAFF), or IL-4) to the MATRIGEL®, regardless of whether the MATRIGEL® is added to the MOS mix or to the culture medium. These strategies also may help to retain the MM cells within the MOS and/or to keep the MOS from attaching to the bottom of the culture well. Given this, the MM MOS can have a cell number of about 50 to about 150 cells per MOS, with MM plasma cells, stromal cells, and immune cells included in the MOS cell population. In general, MOS can be generated immediately from a biopsy, and generally retain similar cell ratios as the patient biopsy. In some cases, the ratio of stromal cells to MM cells in a MOS can be about 6:1 to about 3:1 (e.g., about 5:1 or about 4:1). In some cases, the ratio of immune cells (e.g., macrophages) to MM cells in a MOS can be about 1:50 to about 1:200 (e.g., about 1:100).

Once formed, the MOS can be cryopreserved and/or cultured. In general, cultured MOS can be maintained in suspension, either static (e.g., in a well, vial, or other suitable container) or in motion (e.g., rolling or agitated). The MOS can be cultured using any appropriate techniques. Exemplary techniques can be found in, without limitation, Freshney, Culture of Animal Cells, A Manual of Basic Techniques, 4^{th} ed., Wiley Liss, John Wiley & Sons, 2000; Basic Cell Culture: A Practical Approach, Davis, ed., Oxford University Press, 2002; Animal Cell Culture: A Practical Approach, Masters, ed., 2000; and U.S. Pat. Nos. 5,516,681 and 5,559,022, all of which are incorporated herein by reference in their entirety.

### Labeling MOS

Cells (e.g., tumor cells or non-tumor cells) within MOS, or cells in a biological sample containing dissociated cells that will be used to generate MOS, can be stained with a detectable label that is targeted to a selected cell surface marker. Any appropriate detectable label (e.g., a fluorescent label) can be coupled to dissociated cells or to cells within MOS in culture. In some cases, a pH-sensitive detectable label can be used. An exemplary pH-sensitive label is PHRODO™ (Thermo Fisher), a fluorogenic dye that serves as an intracellular pH indicator and can be used to measure pH in live cells. In particular, PHRODO™ is weakly fluorescent at neutral pH, but becomes increasingly fluorescent as pH decreases (e.g., during phagocytosis). It is to be noted that as an alternative to fluorescent dye conjugated antibodies, any dyes that can stain unfixed cells may be used in the methods described herein, including live or dead (e.g., fixed) cell fluorescent dyes, dyes for staining the cytoskeleton or intracellular organelles, and/or dyes that stain lipid droplets.

Any appropriate means can be used to couple a detectable label to specific cells (e.g., cancer cells, immune cells, fibroblasts, endothelial cells, etc.) in a dissociated biological sample (e.g., a tumor sample that has been dissociated), or to cells within MOS in culture. For example, a detectable label can be conjugated to an antibody or an antibody fragment (e.g., a V_{H}H fragment, also referred to as a NANOBODY®) and the conjugate can then be attached to cells in a dissociated tissue sample or in MOS that have already been formed via the antigen-binding domain of the antibody or antibody fragment. The term “antibody,” as used herein, includes full-length antibody molecules (e.g., IgA-, IgD-, IgE, IgG- or IgM-type, antibodies, including IgG- or IgM-types such as, without limitation, IgG1-, IgG2-, IgG3-, IgG4-, IgM1- and IgM2-types), and also includes antibody-like molecules that contain engineered sub-domains of antibodies or naturally occurring antibody variants. Antibody-like molecules may be single-domain antibodies such as V_{H}-only or V_{L}-only domains derived either from natural sources such as camelids (Muyldermans et al. (2001) Rev. Mol. Biotechnol. 74:277-302) or through in vitro display of libraries from humans, camelids, or other species (Holt et al. (2003) Trends Biotechnol. 21:484-90). In certain embodiments, the polypeptide structure of the antigen binding proteins can be based on antibodies, including, but not limited to, minibodies, synthetic antibodies (sometimes referred to as “antibody mimetics”), human antibodies, antibody fusions (sometimes referred to as “antibody conjugates”), and fragments thereof, respectively.

An “Fv fragment” is the minimum antibody fragment that contains a complete antigen-recognition and -binding site. This region consists of a dimer of one heavy chain variable domain and one light chain variable domain in tight, non-covalent association. It is in this configuration that the three CDR's of each variable domain interact to define an antigen-binding site on the surface of the VH-VL dimer. Collectively, the six CDR's confer antigen-binding specificity to the antibody. However, even a single variable domain (or half of an Fv comprising only three CDR's specific for an antigen) has the ability to recognize and bind the antigen, although usually at a lower affinity than the entire binding site. The “Fab fragment” also contains the constant domain of the light chain and the first constant domain (C_{H}1) of the heavy chain. The “Fab fragment” differs from the “Fab′ fragment” by the addition of a few residues at the carboxy terminus of the heavy chain C_{H}1 domain, including one or more cysteines from the antibody hinge region. The “F(ab′)2 fragment” originally is produced as a pair of “Fab′ fragments” which have hinge cysteines between them. Methods of preparing such antibody fragments can include the use of, for example, papain or pepsin digestion.

Nanobodies are recombinantly expressed antigen binding V_{H}H domains from heavy-chain IgG. Nanobodies typically have a molecular weight of about 12 to 15 kDa, and are one of the smallest naturally occurring antigen binding fragments. These isolated V_{H}H domains retain the ability to bind to antigens.

For further discussion of antibody fragments, see, for example, Bates and Power, Antibodies, 8:28, 2019. In addition, kits that can be used to couple a label (e.g., PHRODO™ to an antibody are commercially available (e.g., the PHRODO™ Deep Red Antibody Labeling Kit from Thermo Fisher Scientific).

The antibodies or antibody fragments used to couple a detectable label to cells (e.g., cancer cells) can be targeted to any appropriate cell surface marker (e.g., a cell surface polypeptide or portion thereof) on the cells. For example, the antigen-binding domain of an antibody or antibody fragment can be targeted to an antigen (e.g., a cancer cell-specific antigen) such as, without limitation, CD38, CD138, CS1, BCMA, CD45, cell adhesion molecule, EpCAM, TROP2, CD3, CD8, PD-1, CD-31, CD33, CD19, FAPalpha, ASGR1, or HER2.

In some cases, the antigen-binding domain of an antibody or antibody fragment can be targeted to a cell-specific antigen such as CD33, which is a marker for AML. In some cases, the antigen-binding domain of an antibody or antibody fragment can be targeted to CD31 (a marker for endothelial cells), CD19 (a marker for B cell lymphoma and leukemias), or HER2 (a marker for breast cancer cells).

In some cases, live cells can be labeled by adding, to medium containing dissociated cells from a tissue sample, an appropriate amount of one or more antibodies or antibody fragments that are each coupled to a detectable label. For example, about 0.005 μg to about 5 μg (e.g., about 0.005 μg to about 0.05 μg, about 0.05 μg to about 0.5 μg, or about 0.5 μg to about 5 μg) of one or more antibody-conjugated detectable markers can be added to a well of a plate (e.g., a 96-well plate) that contains from about 100 to about 100,000 cells (e.g., about 100 to about 500, about 500 to about 1000, about 1000 to about 5000, about 5000 to about 10,000, about 10,000 to about 50,000, or about 50,000 to about 100,000 cells). In some cases, live cells within MOS can be labeled by adding, to MOS that have already been formed and are in culture, an appropriate amount of one or more antibodies or antibody fragments that are each coupled to a detectable label. For example, 0.005 μg to about 5 μg (e.g., about 0.005 μg to about 0.05 μg, about 0.05 μg to about 0.5 μg, or about 0.5 μg to about 5 μg) of an antibody-conjugated detectable marker can be added to a well of a plate (e.g., a 96-well plate) that contains from about 50 to about 400 MOS (e.g., about 50 to about 100, about 100 to about 200, about 200 to about 300, or about 300 to about 400 MOS). It is to be noted that when two or more labeled antibody or antibody fragments targeted to different cellular antigens are used, each antibody or antibody fragment can be targeted to a different antigen. In some cases, the different antigens can be present on different types of cells. Further, each of the two or more antibodies or antibody fragments can be coupled to a different detectable marker, such that differentially labeled cells can be distinguished from each other. The cells or the MOS can be incubated with the detectable marker(s) for any suitable length of time (e.g., about 30 minutes to about 4 hours). In some cases, the cells or MOS can then be washed to remove excess label. When one or more antibody-coupled labels are applied to dissociated cells, the cells can then be used to generate MOS. The MOS, whether they contain cells that were labeled prior to MOS formation or after MOS formation, can then be imaged to detect the label using any appropriate means (e.g., flow cytometry or fluorescence microscopy). In some cases, the MOS can be used in other assays, depending on the cells to which the detectable label is bound.

### Methods for Using Labeled MOS

MOS containing cells (e.g., live cells) that are detectably labeled as described herein can be used in a variety of assays. For example, particular cells (e.g., tumor cells, immune cells, fibroblasts, or endothelial cells) can be labeled before or after they are incorporated into MOS, and the MOS can be imaged to observe where the cells are present, how they interact with other cells within the MOS, etc. In some cases, MOS containing labeled cells can be used to screen various therapeutic agents to predict which one(s) might be effectively and safely used in the patient from whom the tissue sample was taken. For example, MOS can be used in toxicity screens for drugs (e.g., immuno-oncology or “IO” drugs) or other chemical compositions, and to determine whether one or more drug compositions might effectively treat a patient before the patient undergoes drug therapy, based at least in part on the response of the cells within the MOS to the one or more drugs. This can allow for very rapid screening of patients before they would otherwise undergo months of treatment that may not be effective. Methods of using MOS for screening can be automated or manually performed. A general description of screening methods using MOS can be found in U.S. Patent Publication No. 2021/0285054 at, for example, paragraphs [0190] to [0194] and FIG. 17.

In some cases, the methods and materials provided herein can include using MOS containing labeled live cells to assess phagocytosis of cells in the MOS, and/or to evaluate the effect of candidate agents (e.g., therapeutic agents such as drugs) on phagocytosis of the labeled cells. Phagocytosis is a cellular process for ingesting and eliminating particles with a diameter of at least 0.5 μm, such as apoptotic cells, microorganisms, and foreign substances. Phagocytosis is most efficiently carried out by specialized cells, including macrophages, neutrophils, monocytes, dendritic cells, and osteoclasts (referred to as phagocytes), which express receptors that activate signaling pathways resulting in phagocytosis. Other cell types, including, fibroblasts, epithelial cells, and endothelial cells, also can accomplish phagocytosis, but at a lower efficiency. See, e.g., Uribe-Querol and Rosales, Front. Immunol., 11:1066, 2020; doi.org/10.3389/fimmu.2020.01066.

In some cases, PMOS (e.g., MM MOS) containing labeled cells can be used in screening assays to determine the effects of one or more potential therapeutic agents on phagocytosis of labeled cancer cells by phagocytes within the PMOS. For example, a composition to be tested can contain a drug, drug dilution, drug formulation, combination of drugs (e.g., multiple active ingredients), drug forms, drug concentrations, and the like. In some cases, a drug formulation can be a formulation containing a mixture of a drug and one or more inactive ingredients. MOS containing labeled cells also can also be used in longitudinal high throughput screens to identify additional (e.g., improved) labeling reagents for technology and assay development, for example. For example, MOS containing labeled cells can be used in longitudinal studies to identify new functional dyes, to track specific cell populations, and/or to capture dynamic responses longitudinally, such as by following drug effects on specific cell populations over time in a complex mixture of cells from a primary sample that are contained within the MOS.

In some cases, immune cells that are encapsulated in MOS or that are exogenously added to pre-formed MOS can be labeled using the methods described herein, and the label can be detected/measured to assess the behavior of immune cells. For example, labeled anti-CD45 antibodies can be used to stain leukocytes, labeled anti-CD3 antibodies can be used to stain T cells, labeled anti-CD8 antibodies can be used to stain cytotoxic T cells, labeled anti-CD4 antibodies can be used to stain helper T cells, labeled anti-CD56 antibodies can be used to stain NK cells, and labeled anti-CD19 antibodies can be used to stain B cells. In some cases, such labeled antibodies can be added to MOS that encapsulate immune cells and cancer cells. In some cases, such labeled antibodies can be used to stain immune cells that are to be exogenously added to cultures of MOS that encapsulate cancer cells. After staining, the label can be detected and/or measure to track interaction of the immune cells with cancer cells in the MOS. In some cases, the label can be detected/measured to assess the response of the cells to drug treatment (e.g., treatment with an IO drug).

The steps in carrying out an assay provided herein can include, for example, placing MOS containing labeled live cells into wells of a multi-well plate (e.g., a 96-well plate) or a multi-well grid (e.g., a 10,000 micro-well array formed of a 100×100 well grid). The MOS (e.g., gel droplets) can be applied into, or in some cases onto, the microwell arrays and incubated with culture medium. The MOS can be cultured over the course of about 1 to about 14 days. On a selected day (e.g., day 3), the wells can be dosed with one or more candidate phagocytosis-stimulating agents, to examine the effects of the agent(s) on phagocytosis of cancer cells within the MOS. The wells can be imaged (e.g., via fluorescent microscopy) to assess the signal output from the pH-sensitive label and determine whether phagocytosis has occurred and/or whether the level of phagocytosis is affected by one or more of the candidate agents. In some cases, the signal output can be measured over time, in order to assess the rate of phagocytosis (e.g., to determine whether a drug is capable of increasing the level or rate of phagocytosis of tumor cells by phagocytes within MOS from a cancer patient). An increase in the level or rate of phagocytosis after addition of an agent can indicate that the agent would be useful as a therapeutic against cancer cells within the mammal from which the biological sample used to generate the MOS was obtained.

The invention will be further described in the following examples, which do not limit the scope of the invention described in the claims.

### EXAMPLES

### Example 1—Labeling of MOS Containing CRCs

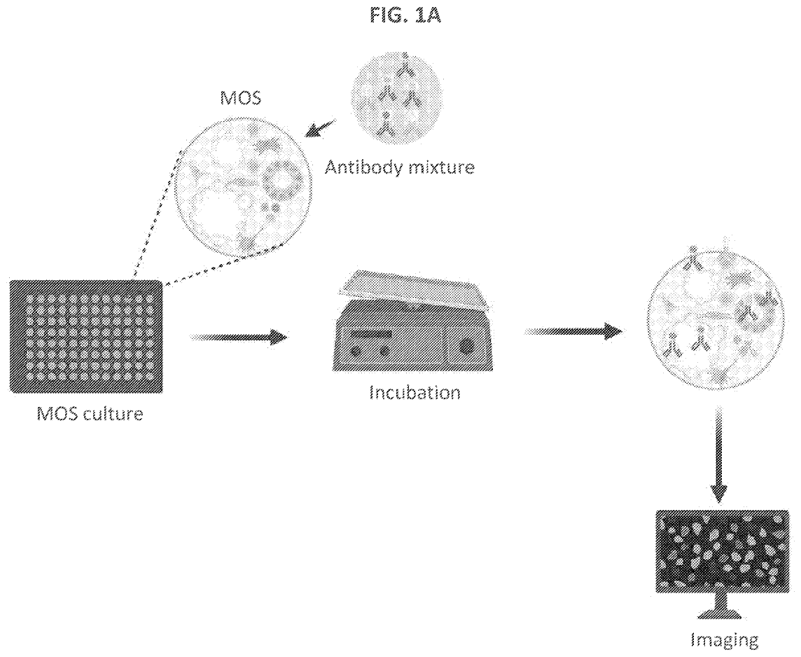

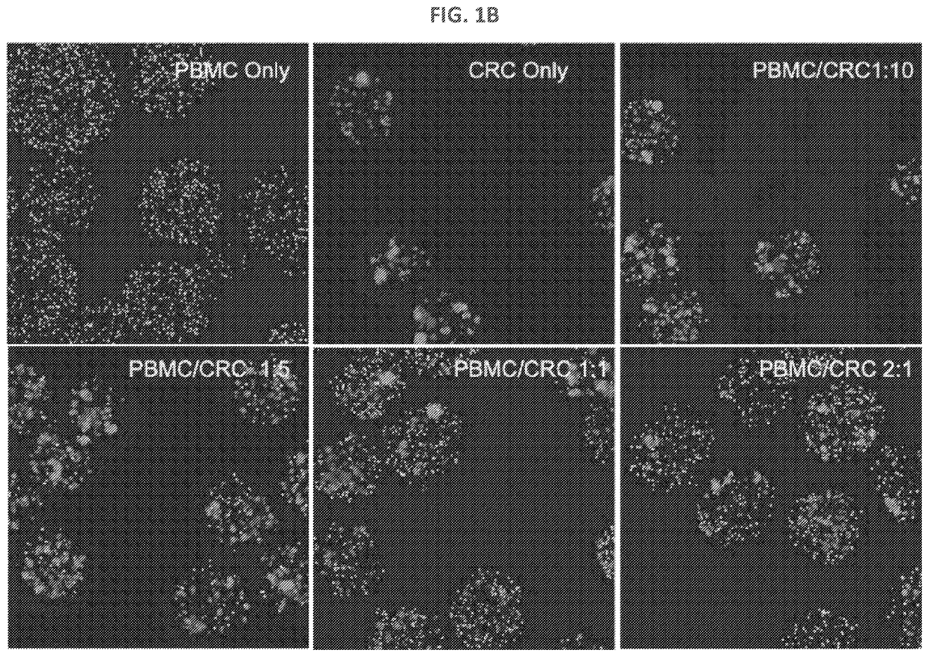

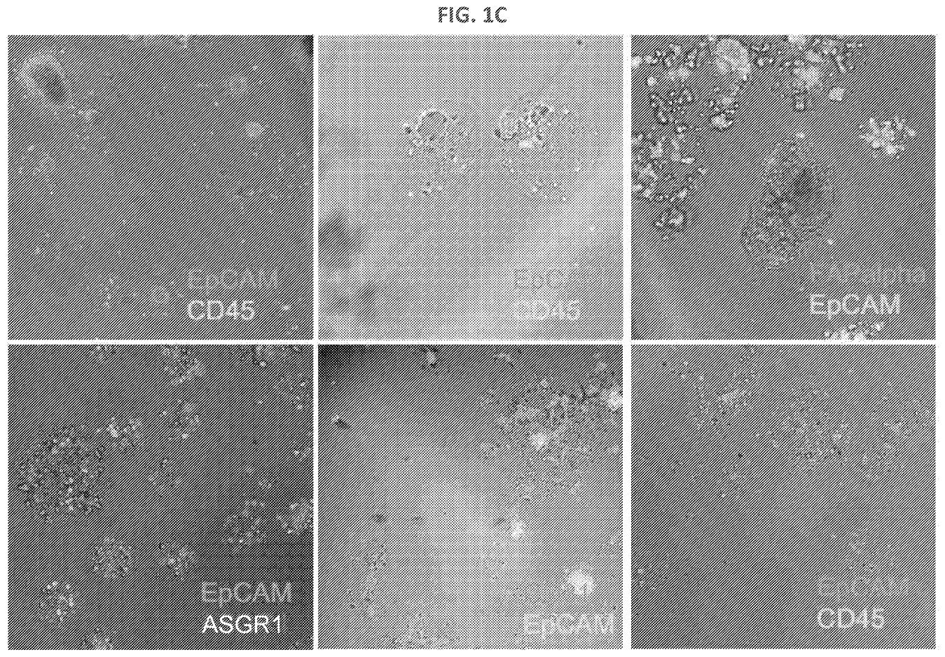

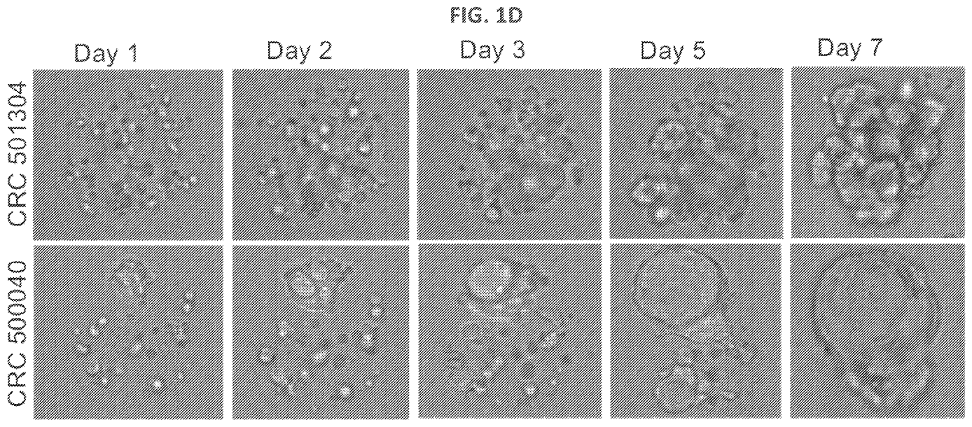

Live MOS were stained according to the method illustrated in FIG. 1A. Different antibodies were conjugated with different colors of fluorescent dyes at ratios of about 1:100 to about 1:2000. The antibody mixture was then added to wells containing MOS generated from CRC cell lines. After incubation for 1-2 hours on a rocker in an incubator, the labeled MOS were imaged using an IMAGEXPRESS® Micro Confocal High-Content Imaging System (IXM; MOLECULAR DEVICES® San Jose, CA) or another suitable imaging platform. Representative images of MOS stained with CD45 (green) and EpCAM (magenta) are shown in FIG. 1B. The top panel of the figure shows, from left to right, MOS that encapsulated PBMC, MOS that encapsulated CRC cells, and MOS that encapsulated both PBMC and CRC cells at a 1:10 ratio. The bottom panel shows, from left to right, MOS that encapsulated both PBMC and CRC cells at a 1:5 ratio, MOS that encapsulated both PBMC and CRC cells at a 1:1 ratio, and MOS that encapsulated both PBMC and CRC cells at a 2:1 ratio. The antibody mixtures (EpCAM and CD45) were added to the wells with different types of MOS, and after staining for about 1 hour, the wells were imaged using IXM. These studies demonstrated labeling of cells within the PBMC population with CD45, and CRC cells with EpCAM. Additional representative images of the antibody-stained MOS are shown in FIG. 1C. The top panel shows, from left to right, MOS that encapsulated CRC and tumor infiltrated lymphocytes, stained with EpCAM and CD45; MOS derived from primary lung tissue, stained with EpCAM and CD45; and a mixed culture MOS that encapsulated cancer-associated fibroblasts or CRC cells, stained with FAPalpha and EpCAM. The bottom panel shows, from left to right, MOS that encapsulated both CRC and hepatocytes, stained with EpCAM and ASGR1; MOS that encapsulated CRC cells, HUVEC, and cancer-associated fibroblasts, stained with CD31 and EpCAM; and MOS that encapsulated primary lung-derived cells, stained with EpCAM and CD45. In addition, FIG. 1D shows representative images of MOS generated from two CRC cell lines (501304 and 500040) stained with EpCAM, where the fluorescence signals from the EpCAM staining were captured using a CELIGO® imaging cytometer (Redwood City, CA). These studies further demonstrated appropriate staining of cells within MOS using differently labeled antibodies targeted to different cellular markers.

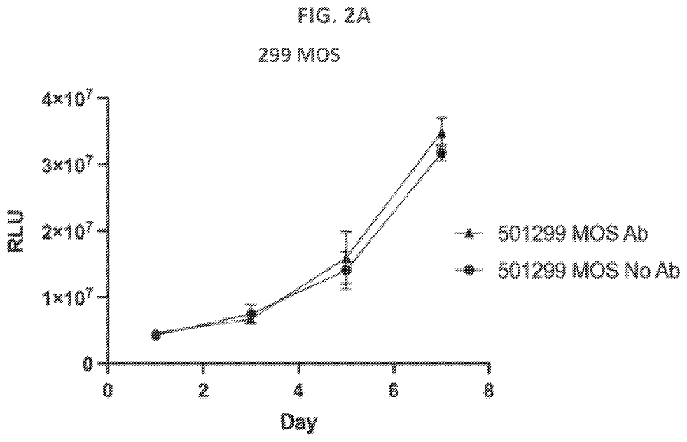

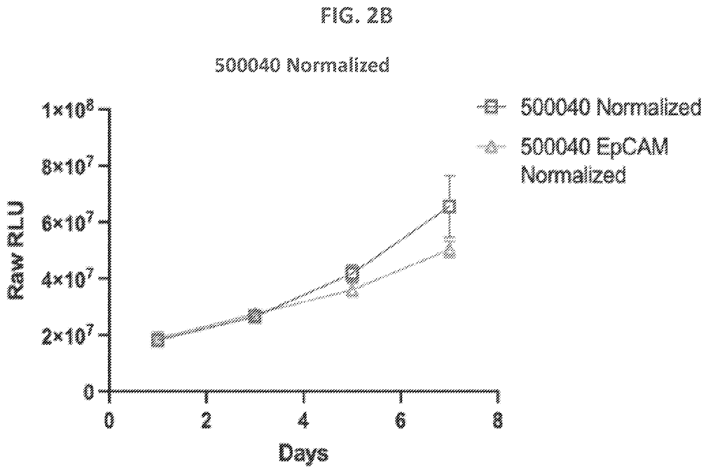

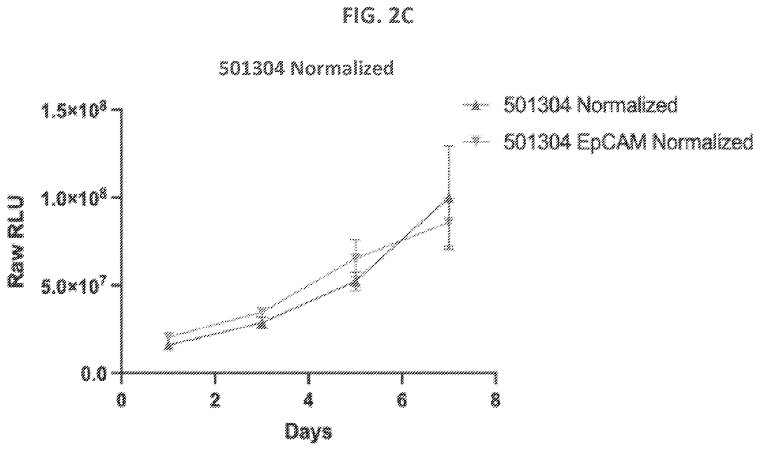

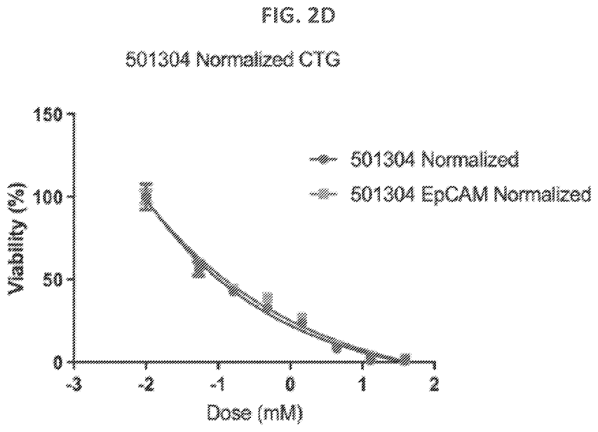

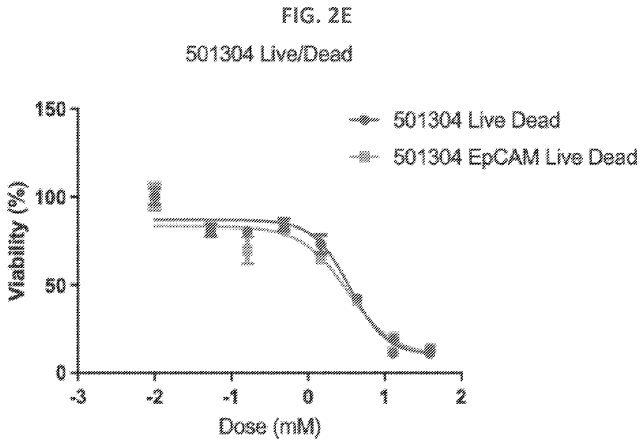

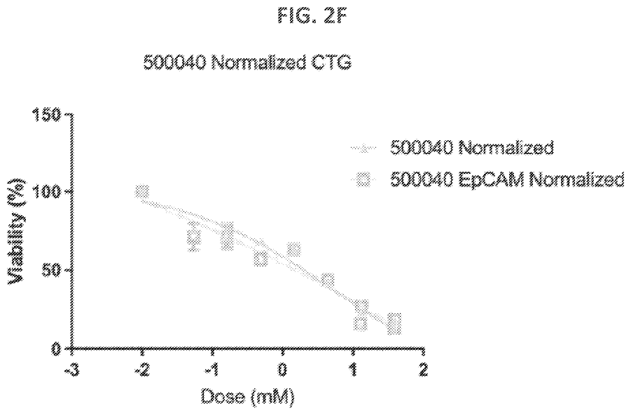

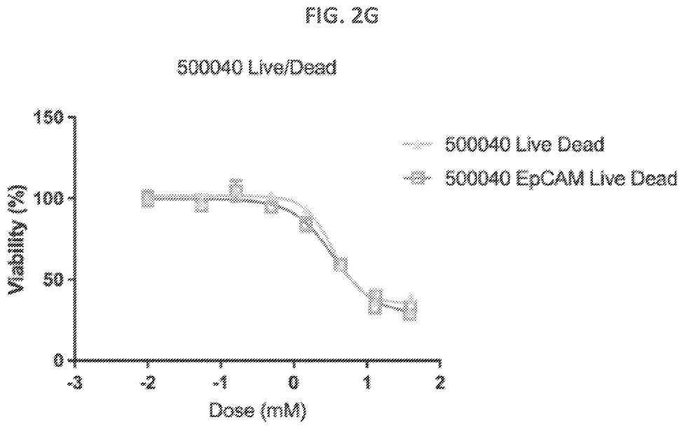

Further studies were conducted to determine how staining of MOS might affect growth or drug responses of the MOS. As shown in FIGS. 2A-2C, there were no obvious growth differences of MOS generated from three different CRC cells when the MOS were or were not stained with EpCAM over 7 days of culture, as measured by CTG assay. Further, no difference in drug response was observed when MOS generated from two different CRC cell lines were or were not stained EpCAM at a dilution of 1:500, followed by treatment with different doses of 5-FU for 3 days. Drug response was measured by CTG assay (FIG. 2D for 501304 cells and FIG. 2F for 500040 cells) or by live/dead cell dye staining (FIG. 2E for 501304 cells and FIG. 2G for 500040 cells).

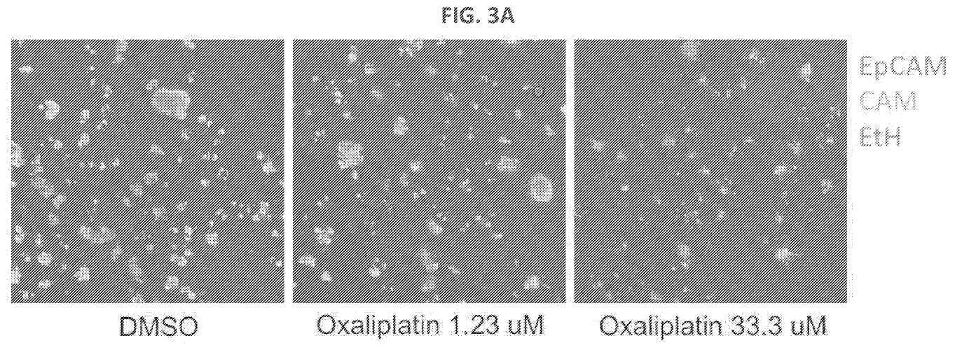

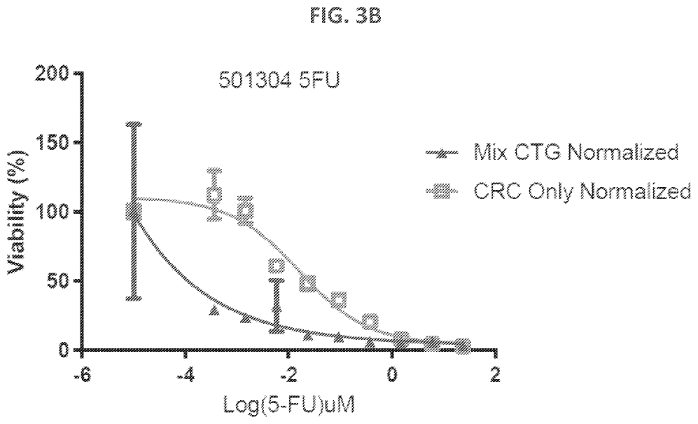

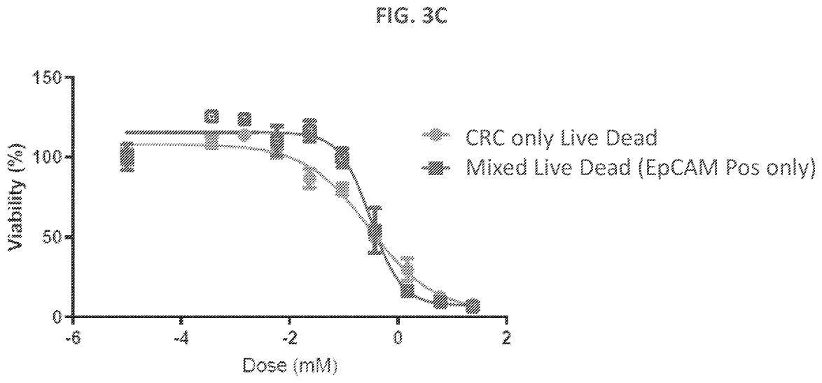

Additional images of stained live MOS are shown in FIG. 3A, which includes representative images of live MOS containing CRC cells co-encapsulated with fibroblasts, endothelial cells, and PBMCs, where the MOS were stained with EpCAM and treated with vehicle or oxaliplatin (1.23 μM or 33.3 μM) for three days, followed by staining with Calcein AM (CAM) and ethidium homodimer (EtH). The higher dose of oxaliplatin resulted in increased staining with EtH and less staining with Calcein AM, indicating a greater degree of killing. When MOS encapsulating only CRC cells or MOS co-encapsulating CRC cells, fibroblasts, endothelial cells, and PBMCs were treated with oxaliplatin, differential responses to the treatment were observed between the CRC-only MOS and the co-encapsulated MOS, as determined by CTG assay (FIG. 3B). However, the drug responses of the CRC-only MOS versus the CRC/non-cancer cell co-encapsulated MOS were much more similar based on live cell dye (CAM): dead cell dye (EtH) ratios from EpCAM positive (FIG. 3C).

### Example 2—Use of Labeled MM MOS in Phagocytosis Assays

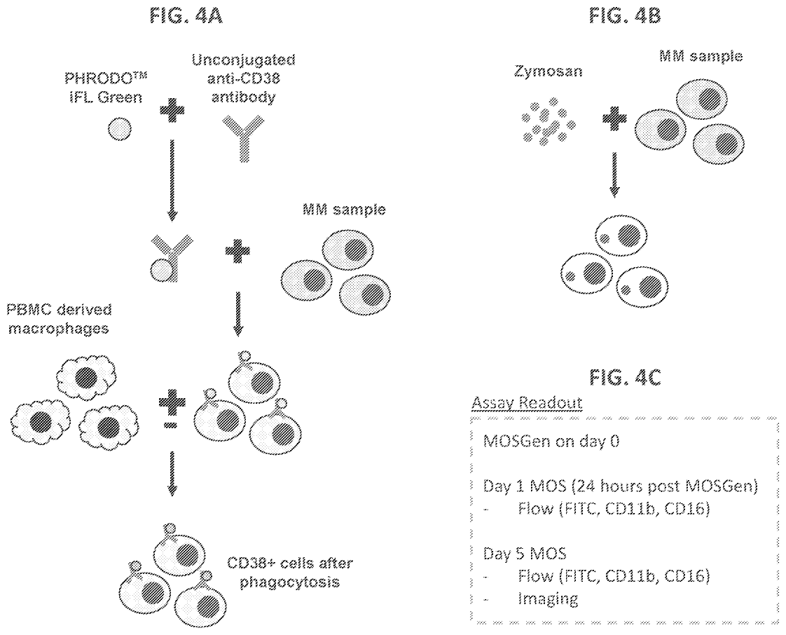

MM MOS containing PHRODO™-labeled myeloid cells were generated as depicted in FIG. 4A. Phagocytosis of the labeled myeloid cells results in a green fluorescent signal that can be detected by flow cytometry. As shown in FIG. 4A, CD38 antibodies were labeled with PHRODO™, MOS were generated from a MM sample and cultured with PHRODO™-labeled CD38 antibody to tag CD38⁺ myeloid cells within the MOS, and the PHRODO™-tagged MOS were cultured with or without PBMC-derived macrophages before imaging (FIG. 4C). Co-culture with macrophages derived from monocytes (MDM macrophages) was included as a positive control to show that phagocytosis was occurring.

MM cells also were labeled with PHRODO™-Zymosan to provide a total measure of phagocytosis, as shown in FIG. 4B. Zymosan was labeled with PHRODO™ green, and the PHRODO™-labeled Zymosan was co-packaged with a MM sample, followed by flow cytometry to assess the level of phagocytosis.

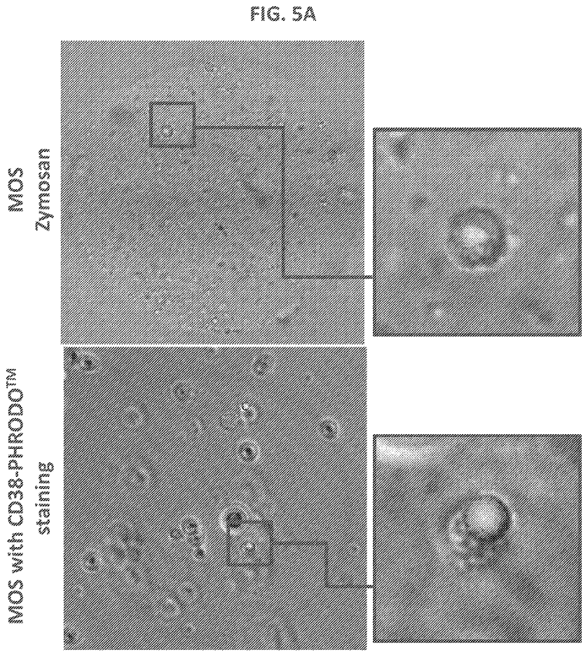

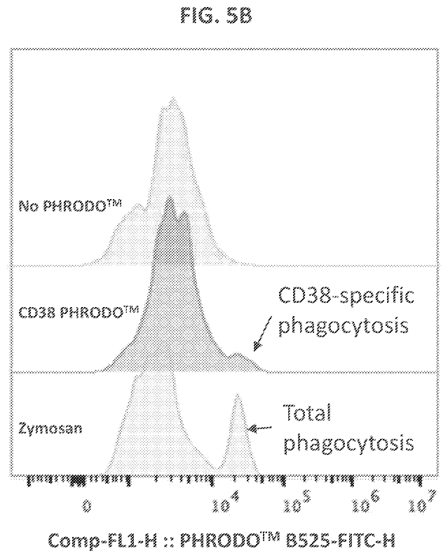

Readouts from the methods depicted in FIGS. 4A and 4B are listed in FIG. 4C. Specifically, at Day 1 of MOS culture (about 24 hours after MOS generation) and at Day 5 of MOS culture, flow cytometry was carried out to measure cells labeled with fluorescein isothiocyanate (FITC), CD11b , and CD16. The MOS also were imaged on Day 5. Representative images are shown in FIG. 5A. MM MOS were cultured with PHRODO™-labeled Zymosan (top) or PHRODO™-labeled anti-CD38 antibodies (bottom). FIG. 5B shows histograms generated by flow cytometry for MM MOS with no labeling (top), PHRODO™-labeled anti-CD38 antibodies (middle), or PHRODO™-labeled Zymosan (bottom). These studies demonstrated that phagocytosis of CD38⁺ cells was detected in MM MOS.

The percentage of phagocytes and CD11b⁺ phagocytes was measured and plotted in the graphs shown in FIGS. 6A and 6B. MOS were generated directly from frozen thawed MM samples, and PHRODO™-labeled antibodies or PHRODO™-labeled Zymosan was added to the culture medium just before MOS generation. Data were collected after 24 hours for (1) unlabeled MM samples, (2) Zymosan-labeled MM samples, (3) PHRODO™-labeled MM samples, and (4) MM samples with PBMC-derived macrophages. The percentage of green cells in live cells for each condition after 24 hours of incubation is plotted in FIG. 6A. The increased percentage of phagocytes observed in the “Tumor” and “Tumor⁺ MDM” samples indicated that phagocytosis occurred in this MM sample. The percentage of CD11b⁺ cells from the green cells of FIG. 6A is plotted in FIG. 6B, showing that almost all of the green cells were CD11b⁺. These studies demonstrated that phagocytosis was detected in Day 1 MM MOS, that the majority of phagocytic cells were CD11b positive, and that adding MDM increased the percentage of phagocytosis positive events (less than 5% of the total cells were derived macrophages.

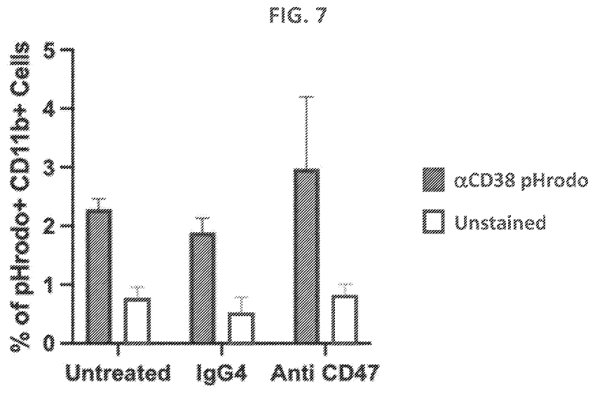

Additional studies to determine the percentage of phagocytes and, more specifically, CD11b positive phagocytes, were conducted after 5 days of culture, and the results are plotted in FIG. 7. MOS were generated directly from frozen thawed MM sample and cultured for 5 days. Anti-CD47 or IgG4 control was added to the culture media beginning at Day 0 (the day of MOS generation). PHRODO™-labeled antibodies were added to the culture medium on Day 5, and data were collected 24 hours later. The conditions used untreated, IgG4 added on Day 5 after MOS generation, and 10 μg/ml anti-CD47 added on Day 5 after MOS generation. The percentage of green CD11b⁺ cells in live cells for each condition after 24 hours of incubation is plotted in FIG. 7. All conditions were in MM samples co-packaged with PBMC derived macrophages. The increased percentage of phagocytosis seen in “untreated” demonstrated that phagocytosis occurred, while the increased percentage of phagocytosis in the anti-CD47 samples suggested that anti-CD47 induced more phagocytosis. These studies demonstrated that phagocytosis was detected in Day 5 MM MOS, that adding MDM increased the percentage of phagocytosis positive events, and that anti-CD47 induced phagocytosis.

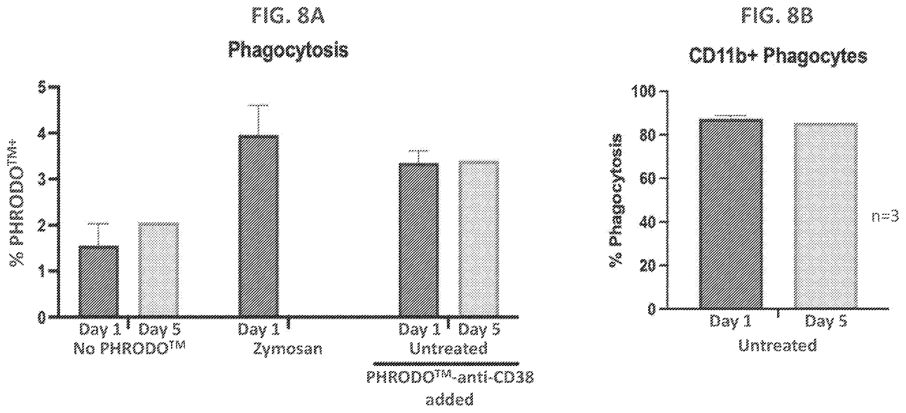

A frozen MM sample from a second patient was used to validate the PHRODO™-antiCD38 assay. All data were generated by flow cytometry. MOS were generated directly from frozen thawed MM samples, and PHRODO™-labeled antibodies were added to the culture medium either immediately after MOS generation on Day 0 or on Day 5 of MOS culture. Data were collected 24 hours later. The conditions used were: (1) no PHRODO™-labeled antibody added; (2) MM co-packaged with PHRODO™-labeled Zymosan; and (3) untreated (no drug added). The percentage of green cells in live cells for each condition after 24 hours of incubation is plotted in FIG. 8A. The increased percentage of green cells observed in the “Untreated” sample demonstrated that phagocytosis had occurred in this MM sample. FIG. 8B is a graph plotting gating of CD11b⁺ cells from the green cells of FIG. 8A, indicating that almost all of the green cells were CD11b⁺. Consistent with the work described above, these studies demonstrated that phagocytosis is detected in Day 1 and Day 5 MM MOS, and that the majority of phagocytic cells were CD11b positive.

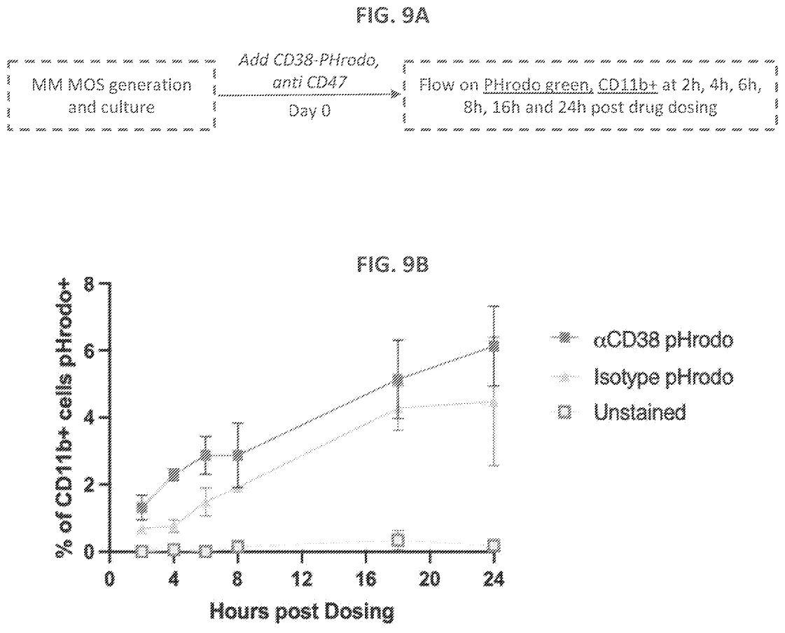

In further studies, a time course assay was conducted in which PHRODO™-labeled CD38 antibody was used to tag MM myeloid cells in MOS, and an anti-CD47 antibody was used to treat the MOS, followed by flow cytometry at 2, 4, 6, 8, 16, and 24 hours after dosing (FIG. 9A). This assay demonstrated that a higher percentage of CD11b⁺ cells in MOS stained with PHRODO™-anti-CD38 were PHRODO™⁺ than in MOS stained with isotype PHRODO™ (FIG. 9B).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
labeling a population of cells within Micro-OrganoSpheres (MOS) with a detectable label, wherein at least a portion of the cells within the population comprise the detectable label.

2. The method of claim 1, wherein the labeling comprises adding the detectable label to already formed MOS.

3. The method of claim 1, wherein the labeling comprises adding the detectable label to the population of cells before the MOS are formed.

4. The method of claim 1, wherein the portion of cells comprise cancer cells, immune cells, fibroblasts, endothelial cells, stromal cells, or epithelial cells.

5. The method of claim 1, wherein the MOS comprise multiple myeloma cells.

6. The method of claim 1, wherein the detectable label is a fluorescent dye, or a pH-sensitive label.

7. (canceled)

8. The method of claim 1, wherein the detectable label is attached to the cells via an antibody or antibody fragment, wherein the antibody or antibody fragment comprises an antigen binding region that interacts with an epitope on the cells.

9-10. (canceled)

11. The method of claim 1, wherein the label is a pH-sensitive label, and wherein the method further comprises measuring the level of signal over time to determine a rate or extent of phagocytosis, or wherein the method further comprises detecting the presence or absence of a signal output from the pH-sensitive detectable label, wherein the presence of the signal indicates phagocytosis of cells within the population, and wherein the absence of the signal indicates a lack of phagocytosis of cells within the population.

12. (canceled)

13. A method comprising:
providing MOS comprising a population of cells, wherein at least a portion of the cells within the population comprise a pH-sensitive detectable label,
measuring a first level of signal from the pH-sensitive detectable label, wherein the first level of signal is directly correlated to a level of phagocytosis of the cells within the portion of cells,
contacting the MOS with a candidate agent,
measuring a second level of signal from the pH-sensitive detectable label, wherein the second level of signal is directly correlated to a level of phagocytosis of cells within the portion of cells after the contacting, and
comparing the first measured level of signal to the second measured level of signal and, when the second measured level of signal is increased compared to the first measured level of signal, identifying the candidate agent as being a phagocytosis-enhancing agent for the population of cells.

14. The method of claim 13, wherein the portion of cells comprise cancer cells.

15. The method of claim 13, wherein the MOS comprise multiple myeloma cells.

16. (canceled)

17. The method of claim 13, wherein the pH-sensitive detectable label is attached to the cells via an antibody or antibody fragment, wherein the antibody or antibody fragment comprises an antigen binding region that interacts with an epitope on the cells.

18-20. (canceled)

21. A method for treating a mammal, the method comprising:
providing MOS comprising a population of cells from the mammal, wherein at least a portion of the cells within the population comprise a pH-sensitive detectable label,
measuring a first level of signal from the pH-sensitive detectable label, wherein the first level of signal is directly correlated to a level of phagocytosis of the cells within the portion of cells,
contacting the MOS with a candidate agent,
measuring a second level of signal from the pH-sensitive detectable label, wherein the second level of signal is directly correlated to a level of phagocytosis of cells within the portion of cells after the contacting,
determining that the second measured level of signal is increased compared to the first measured level of signal, and
administering the candidate agent to the mammal.

22. The method of claim 21, wherein the plurality of cells comprise cancer cells.

23. The method of claim 21, wherein the MOS comprise multiple myeloma cells.

24. The method of claim 21, wherein the pH-sensitive label is a fluorescent dye.

25. The method of claim 21, wherein the pH-sensitive detectable label is attached to the cells via an antibody or antibody fragment, wherein the antibody or antibody fragment comprises an antigen binding region that interacts with an epitope on the cells.

26-28. (canceled)

29. A method for treating a mammal, the method comprising administering a therapeutic agent to a mammal identified as having cells that undergo an increased level of phagocytosis when contacted with the therapeutic agent, wherein the mammal was identified by:
providing MOS comprising a population of cells from the mammal, wherein at least a portion of the cells within the population comprise a pH-sensitive detectable label,
measuring a first level of signal from the pH-sensitive detectable label, wherein the first level of signal is directly correlated to a level of phagocytosis of the cells within the portion of cells,
contacting the MOS with a candidate agent,
measuring a second level of signal from the pH-sensitive detectable label, wherein the second level of signal is directly correlated to a level of phagocytosis of cells within the portion of cells after the contacting, and
determining that the second measured level of signal is increased compared to the first measured level of signal.

30. The method of claim 29, wherein the plurality of cells comprise cancer cells.

31. The method of claim 29, wherein the MOS comprise multiple myeloma cells.

32. (canceled)

33. The method of claim 29, wherein the pH-sensitive detectable label is attached to the cells via an antibody or antibody fragment, wherein the antibody or antibody fragment comprises an antigen binding region that interacts with an epitope on the cells.

34-36. (canceled)
